(19) United States
(12) Patent Application Publication
(10) Pub. No.: US 2026/0273138 A1
(43) Pub. Date: Sep. 17, 2026

(54) EPITHELIAL CELL TUBE FOR TRACHEAL TRANSPLANTATION

(71) Applicant: SEOUL NATIONAL UNIVERSITY R&DB FOUNDATION, Seoul (KR)

(72) Inventors: HYUN WOO SHIN, Seoul (KR); KHALMURATOVA ROZA, Seoul (KR)

(21) Appl. No.: 19/667,818

(22) Filed: May 5, 2026

Related U.S. Application Data
(62) Division of application No. 17/907,837, filed on Jun. 17, 2022.

(30) Foreign Application Priority Data
Oct. 13, 2021 (KR) 10-2021-0135977

(51) Int. Cl. A61L 27/38; A61L 27/36

(52) U.S. Cl. CPC A61L 27/3813; A61L 27/3691; A61L 27/3882; A61L 2430/22

(57) ABSTRACT

In a method for preparation of an epithelial cell tube, epithelial cells are cultured at an air-liquid interface cell culture system to obtain differentiated epithelial cells, the differentiated epithelial cells as a layer are transferred and adhered to a planar support so that cilia of the epithelial cells face a lumen side, and both ends of the planar support are linked to each other so that the layer of epithelial cells faces an outer peripheral surface of the planar support, resulting in forming the epithelial cell tube comprising a columnar support and the layer of differentiated epithelial cells adhered on the columnar support.

### CROSS REFERENCE TO RELATED APPLICATIONS AND CLAIM OF PRIORITY

This application is a divisional application of US Patent Application No. 17/907,837 filed on Aug. 29, 2022, which is a National Stage entry from International Application No. PCT/KR2022/008633, filed Jun. 17, 2022, which claims priority to the benefit of Korean Patent Application No. 10-2021-0135977 filed in the Korean Intellectual Property Office on Oct. 13, 2021, the entire contents of which are incorporated herein by reference.

### BACKGROUND

### 1. Background Art

The present invention relates to an epithelial cell tube for tracheal transplantation and a method for preparation thereof.

### 2. Technical Field

With noticeable development of medical technique, organ transplantation techniques to replace an organ, which is difficult to further treat, with another one of other person, have been developed. Subjects able to be transplanted now include even a heart in addition to skin, cornea and kidneys, and the progress after surgery has also significantly improved compared to the past. However, due to a problem in that the number of transplant donors is so much smaller than the number of recipients, immunorejection, and the like, real cases of performing organ transplantation operation or successful cases of the operation are insufficient.

Therefore, a technique for transplantation of artificial substitutes or tissues which were prepared by culturing recipient’s own cells is currently attracting attention. Although specific examples thereof include artificial skin and cultured skin, the artificial skin formed using synthetic polymer may possibly cause immunorejection, hence entailing a limitation of incompatibility as a transplant skin. On the other hand, for the cultured skin, this is obtained by extracting cells from a recipient’s own normal skin and then culturing the same until it reaches a desirable size, thereby not causing immunorejection to foreign materials.

Culturing cells collected from the recipient is conducted on a glass surface or the surface of a synthetic polymer coated with a specific material. Thereafter, the cultured product is subjected to a process of separating and recovering the cultured cells from the above surface by treating the same using a protease such as trypsin or chemicals. However, such a treatment using the protease or chemicals increases the likelihood of containing impurities in the product, and also entails a problem in which the cells are modified or damaged due to a chemical treatment to cause impairment in inherent functions of the cell or the like. Therefore, researches to overcome the problems as described above are now contiguously proceeding.

The present invention relates to a transplant structure including airway epithelial cells, wherein the epithelial cells are cells to cover the airway and serve as a kind of barrier to protect the respiratory organ, and play a significant role of removing foreign materials through ciliary movement or the like. Airway injury occurs due to various causes such as wound, tumor excision, chronic inflammatory airway disorder, congenital cause, etc., in particular, extensive damage to airway cells may cause partial or complete obstruction of the airway through airway fibrosis, hence resulting in life threatening complications such as demanding repeated surgeries.

The present invention relates to an epithelial cell tube which can regenerate an airway by transplanting normal airway epitheliums in an injured airway portion or basically eliminate a fibrosis process due to damage to epithelial cells, as well as a method for preparation thereof.

### SUMMARY

The present invention relates to an epithelial cell tube for tracheal transplantation and a method for preparation thereof, in particular, an object of the present invention is to provide a method for preparation of an epithelial cell tube which includes culturing epithelial cells in a support so that cilia of the epithelial cells face the lumen side, and then, differentiating the same.

1. An epithelial cell tube, comprising a whole layer of epithelial cells positioned on an outer peripheral surface of a columnar support, wherein the epithelial cells are oriented so that cilia of the epithelial cells face a lumen side.

2. The epithelial cell tube according to the above 1, wherein the whole layer of epithelial cells is formed by adhering a whole layer of the cultured epithelial cells to a planar support through electrostatic attraction.

3. The epithelial cell tube according to the above 1, wherein the tube is formed by linking both ends of the planar support each other including the whole epithelial layer.

4. A method for preparation of an epithelial cell tube, which includes: transcription of a whole layer of epithelial cells to a planar support so that cilia of the epithelial cells face a lumen side; and linking both ends of the planar support each other so that the whole layer of epithelial cells faces an outer peripheral surface of the planar support.

5. The method according to the above 4, wherein the whole layer of epithelial cells is cultured and differentiated at an air-liquid interface.

6. The method according to the above 5, wherein the culturing is performed by bringing cilia of epithelial cells into contact with air while the bottom of the epithelial cells is exposed to a culture medium.

7. The method according to the above 4, further comprising culturing the epithelial cell tube, in which both ends of the support are linked together.

The epithelial cell tube produced by the preparation method according to the present invention is applied to an injured portion, therefore, may provide an epithelial cell layer which is engrafted in tissues so as to induce reduction of stenosis of trachea and inflammation.

### BRIEF DESCRIPTION OF THE DRAWINGS

FIG. 1 is a schematic view of an experimental design, including: culturing in an air-liquid interface (ALI) cell culture system to induce completely differentiated epitheliums; separating the differentiated airway epitheliums; culturing in ALI culture medium before transplantation; and transplanting a membrane including the airway epitheliums adhered thereto in a mouse trachea.

FIGS. 2A and 2B illustrate a transplanting process of an orthotropic organ that transplants the trachea of a male C57BL/6 mouse in a syngeneic mouse, specifically, wherein a graft is inserted between the tracheas of a recipient.

FIGS. 3A-3C illustrate a surgery process of removing a donor airway epithelial layer by a brushing skill, specifically, illustrate results of H&E staining a ciliated epithelial layer without damage and de-epithelialized groups.

FIGS. 4A-4H illustrate results of confirming that the transplantation of human airway epithelia can improve the survival of animal, specifically, illustrate results of H&E staining on day 14 after transplantation, measurement of a diameter of the tracheal lumen, images of trachea segments stained with STEM101, E-cadherin and Ki-67, respectively, and a graph showing quantification of the numbers of neutrophils and macrophages per HPF, respectively.

FIGS. 5A-5H illustrate images of H&E stained trachea segments on day 7 after transplantation, assessment of survival of animals after tracheal transplantation on day 7, measurement of a diameter of the tracheal lumen, results of immuno-histochemical staining trachea sections, and results of calculating the numbers of neutrophils, macrophages, STEM101 positive cells and Ki-67 positive cells, respectively, per HPF by groups.

FIGS. 6A-6E illustrate results of inhibiting fibrosis by the transplantation of human airway epithelia, specifically, illustrate histological observation of p63 positive cells, cilia and secretion on day 14, results of quantification of the numbers of p63-positive cells, goblet cells and cilia cells, respectively, per HPF by groups, and the percentage of collagen intensity by groups on day 14.

FIGS. 7A-7D illustrate results of inhibiting fibrosis by the transplantation of human airway epithelia, specifically, illustrate histological observation of p63 positive cells, cilia and secretion on day 7, and results of quantification of the numbers of p63-positive cells, goblet cells and cilia cells, respectively, per HPF by groups.

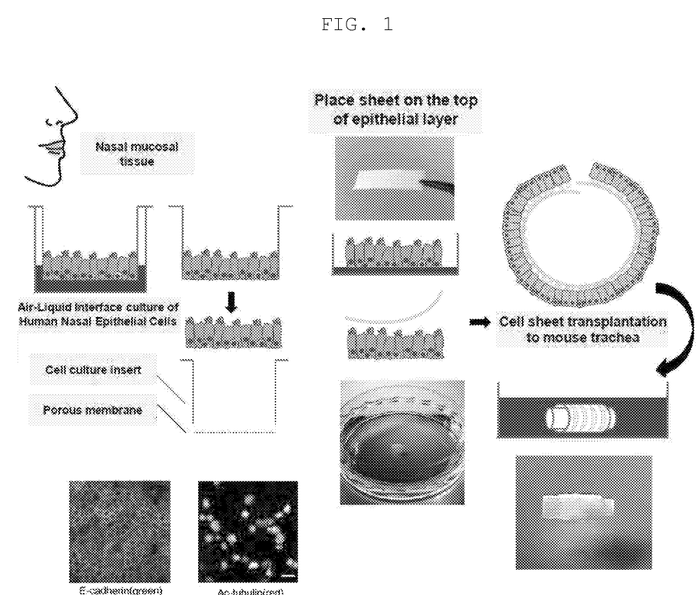

### DETAILED DESCRIPTION

Hereinafter, the present invention will be described in detail.

The present invention may provide an epithelial cell tube including a whole layer of epithelial cells (“whole epithelial layer”) positioned on an outer peripheral surface of a columnar support.

The epithelial cells refer to cells that construct tissues to cover an inner surface of a tubular organ as well as the body surface or body cavity of an animal, for example, may include epithelial cells derived from normal tissues corresponding to an injured portion, but types of the epithelial cells are not particularly limited. For example, if the injured portion is a trachea, the above epithelial cells are preferably airway epithelial cells, but they are not limited thereto.

The epithelial cells are closely adjoined and are combined together through different cell junctions, wherein each cell has polarity, and the surface of the epithelial cell may be divided into an apical surface in contact with the lumen, a lateral surface in contact with neighboring cells, and a bottom surface in contact with a basal lamina. Therefore, the whole epithelial layer means a group of epithelial cells including all of the apical surface, the lateral surface and the bottom surface. Herein, the apical surface of the epithelial cell refers to a surface at which the epithelium and the lumen are in contact with each other, and some epithelial cells have a protruding structure such as a microvillus or a cilium at the apical surface.

In order to restore damaged tissues into a state similar to a tissue lumen and to increase a tissue engraftment rate, the apical surface of the epithelial cell, that is, the cilium may be oriented so as to face the inside, briefly, the support side, but it is not limited thereto.

The epithelial cells may be obtained by any conventional method known in the art such as treatment of epithelial tissues with enzymes, physical separation, etc. For example, in addition to treatment with trypsin or protease, the epithelial cells may also be obtained by scraping the epithelial tissue. Otherwise, the epithelial cells may be differentiated from induced pluripotent stem cells (iPSC) which are derived from skin cells or fibrocytes, or after preparing epithelial tissue organoids, may be obtained therefrom, however, they are not limited thereto.

The epithelial tissue may be composed of single-layered columnar epithelial cells existing in the inner wall of a digestive organ, the bronchial lumen, the nasal cavity, etc., specifically, in the present invention, the epithelial tissue may be the nasal mucosa, but it is not limited thereto.

The epithelial cell tube is formed by adhering the whole epithelial layer to a support. In fact, the whole epithelial layer may be adhered to the support by any conventional method known in the art using the tendency of cells to be grown while being adhered to an object. Specifically, according to the present invention, the epithelial cells may be adhered to the support through electrostatic attraction, but it is not limited thereto.

The support is used for arranging the epithelial cells in a three-dimensional tube shape, and may cause inflammation depending on materials of the support, which in turn may be required to be removed before or after transplantation. The materials used herein may include a variety of materials which are presently used or will be developed in the future, for example, polyvinylidene difluoride (PVDF), polypropylene, polyethylene, cellulose or derivatives thereof, chitin, chitosan, collagen or urethane, in addition to, three-dimensional scaffold materials prepared using 3D-printing techniques, but they are not limited thereto.

The epithelial cell tube may be prepared by rolling the support in a columnar shape and then linking both ends thereof each other.

Further, the present invention may provide a method for preparation of an epithelial cell tube.

The preparation method described above may include: transcription of a whole epithelial layer to a planar support so that cilia of epithelial cells face a lumen side (inside); and linking both ends of the planar support each other so that the whole epithelial layer faces an outer peripheral surface of the support.

Details for the whole epithelial layer and the support are the same as described above.

The transcription step is performed only by adhering the epithelial cell to the support so as to be arranged three-dimensionally, and it is not limited in terms of specific methods for transcription. Specifically, according to the present invention, the transcription may be performed in such a way that cilia of the epithelial cells, that is, the apical surface is adhered to the support side through electrostatic attraction, but it is not limited thereto.

The step of linking the both ends of the support each other may be performed by arranging both ends of the support, to which the whole epithelial layer was adhered, so as to be overlapped with each other. Thereafter, the epithelial cells adhered to the inner side of the support at the overlapped both ends may become extinct.

The whole epithelial layer may be cultured by any conventional culturing method with a culture medium under desired culturing conditions, which are all known in the art. Herein, the culturing condition and method are not particularly limited so long as the obtained cells can be differentiated while maintaining the original function thereof by the above culturing method under the above conditions. For example, the whole epithelial layer may be cultured by adhesive culture, suspension culture or a three-dimensional culturing method. In an aspect that some of the cells are exposed to atmosphere and can be efficiently differentiated, preferably, culturing and differentiation at an air-liquid interface as one of the three-dimensional culturing methods are implemented.

As such, an air-liquid interface (ALI) culturing method is a technique for culturing and differentiating cells which have features similar to those of stem cells or nasal epithelial cells in terms of morphology and functions. In fact, the culturing of epithelial cells at the air-liquid interface may induce differentiation of the epithelial cells into cilia cells, specifically, the cilia of epithelial cells contact air and the bottom surface thereof may be cultured while being exposed to a culture medium. In this regard, the culturing method is not particularly limited so long as functional cells to differentiate the epithelial cells can be cultured.

The preparation method of an epithelial cell tube according to the present invention may further include additional culturing step after the linking of both ends of the support each other.

The additional culturing step is for increasing an engraftment rate of the epithelial cell tube in damaged tissues, and may be implemented before transplanting the tube in the damaged tissues. Herein, a culturing time is not limited to any specific time so long as the cells adhered to the support can be stabilized during the culturing time. For example, the culturing time may range from 1 to 72 hours, 2 to 48 hours, 3 to 24 hours, 4 to 12 hours, 2 to 24 hours, or 3 to 36 hours, but it is not limited thereto. The culturing method and culturing conditions may be implemented by the above-described methods or any conventional method known in the art.

Further, the present invention may provide a tissue regeneration method, which includes transplanting an epithelial cell tube in an injured portion of a subject, wherein a whole epithelial layer positioned on an outer peripheral surface of a columnar support is included, and the epithelial cells are arranged so that cilia thereof face the inside.

When the epithelial cell tube is transplanted in an injured portion of a subject, the injured portion may be reinforced or substituted with the tube, thereby enabling regeneration of the damaged tissues. Accordingly, for example, the epithelial cell tube of the present invention can be used to treat wounds of organ epithelial cells, or stenosis, inflammation, fibrosis, etc. due to the wounds. The organ may be specifically a respiratory organ or a digestive organ.

The subject may refer to all animals including human. For example, mammals including the human may be included, but they are not limited thereto.

The damage may include damages caused by chronic or acute tissue injury, wounds, excision, inflammatory disease, congenital disease, etc.

The epithelial cells may include autocytes, homocytes or heterocytes of the subject under transplantation. The transplantation may include auto-transplantation, homo-transplantation or hetero-transplantation (i.e., xeno-transplantation) depending on the origin of the epithelial cells.

The transplantation may include providing, adhering or fixing the tube to the injured portion.

The injured portion is preferably the inside of a tubular tissue. For example, the injured portion may be the bronchial lumen, the inner wall of the digestive organ, etc., specifically, an airway epithelial tissue.

The tube has a rolled structure and may be efficiently adhered to the inside of a tubular organ due to self-expandable nature.

Specifically, when the injured portion is an airway epithelial tissue, the above transplant operation method may be used as a method for protecting or treating an injury occurring in the airway, tumor excision, chronic inflammatory airway disease, airway obstruction, nose polyp, tracheal granuloma, pulmonary fibrosis, etc.

Details for the whole epithelial layer, the support and the tube are the same as described above.

The above method may include: transcription of the whole epithelial layer to a planar support so that cilia of epithelial cells face the inside; and linking both ends of the planar support each other so that the whole epithelial layer faces an outer peripheral surface of the support.

Details for the transcription and the linking steps are the same as described above.

The above method may further include additional culturing step in order to increase the engraftment rate at the injured portion after linking the both ends of the support each other.

The above method may further include removing the support in order to prevent an occurrence of inflammation depending on materials of the support.

The removal of the support or a removal time may be suitably selected by those skilled in the art in consideration of the materials of the support or a degree for inflammation of the subject.

Hereinafter, the present invention will be described in detail by means of examples. The following examples are provided for illustrating the present invention, and contents of the present invention are not limited to the following examples.

### EXAMPLES

### 1. Experimental method

### 1. Animal experiments

54 C57BL/6 male mice (5-weeks old, 24-25 g) were purchased from Central Lab, Animal Inc., and all animals were housed in a specific facility without pathogens and freely fed with water and food. All experiments in relation to the animals were approved by the Institutional Animal Care and Use Committee (IACUC) of the Institute of Laboratory Animal Resources in Seoul National University, and also have been performed in compliance with the government guide and the international guidance.

### 2. Separation of human airway epithelial cells and air-liquid interface (ALI) cell culture system

After culturing a nasal mucosa of a subject in a control along with 0.1% protease for 1 hour, epithelial cells were collected by scraping the cultured membrane. After washing with DMEM (Dulbecco modified Eagle medium), the cells were maintained on a basic medium for growth of bronchial epithelial cells (Lonza, Basel, Switzerland, cc-3171, cc4175). The nasal epithelial cells were dispensed on a polyester transwell insert having a size of 0.4 μm and 0.33 cm² (Costar, Corning, NY) at a cell density of 5 × 10⁴ cells/well. ALI culture medium was prepared of 1:1 mixture of DMEM medium, to which antibiotics (1% penicillin and streptomycin) and anti-fungal agent (0.2% amphotericin B, Life Technologies, Grand Island, New York) are added, as well as a bronchial and epithelial cell growth medium Bullet kit (Lonza). ALI culture was maintained for 14 days in order to induce completely differentiated pseudostratified epithelia.

### 3. Donor procedure

18 C57BL/6 mice were used as recipient animals. The recipients were administered euthanasia with intra-peritoneal (IP) injection of Avertin (400 mg/kg, Sigma-Aldrich). In order to expose whole tracheas, midline cervical incision was conducted. The tracheas below cricoid cartilage at the end of a branch point were incised and collected. A whole length of the tracheas (about 10 cartilage rings) was divided into two parts at four (4) cartilage rings. Then, the prepared specimen was stored in 1 × PBS for about 5 minutes, followed by storage overnight in ALI culture medium. All surgical procedures were implemented with assistance of a dissecting microscope under aseptic conditions.

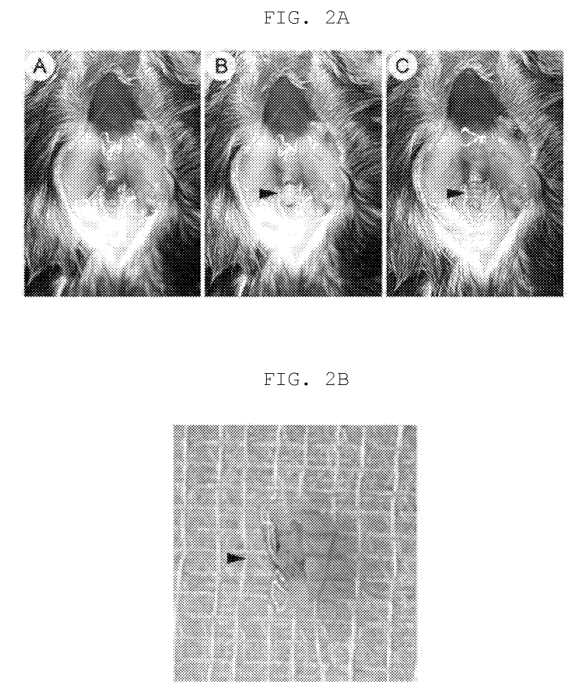

Three major experimental groups (FIGS. 2A and 2B):

Group 1. The trachea including no-injured epithelia, a normal trachea including pseudostratified columnar epithelia used as a control.

Group 2. De-epithelialization of the mouse trachea. Trachea segments were de-epithelialized by a specific brushing skill (interdental toothbrush, 0.4 mm (SSSS), Everden). Efficiency of the de-epithelialization using the brushing skill was verified by H&E staining.

Group 3. Transplantation of human airway epithelia into the de-epithelialized trachea. Before transplanting the human airway epithelia, the trachea segments were de-epithelialized by the brushing skill.

Delivery of airway epithelial sheet with preserved polarity

After removing a porous membrane from the ALI insert, the differentiated airway epithelia were gently separated using a thin glass. With forceps, UpCell support film (ThermoScientific) was disposed on an upper end of the airway epithelia. The film having the airway epithelia adhered thereto was prepared in a three-dimensional tube shape and then delivered to the mouse trachea, followed by fixing the same to the lumen thereof. The grafted epithelia were polarized so that the peak surface faces the lumen side while the bottom surface faces the de-epithelialized trachea. The specimen was maintained overnight in ALI culture medium at 37° C.

### 4. Recipient procedure - transplantation of orthotropic trachea

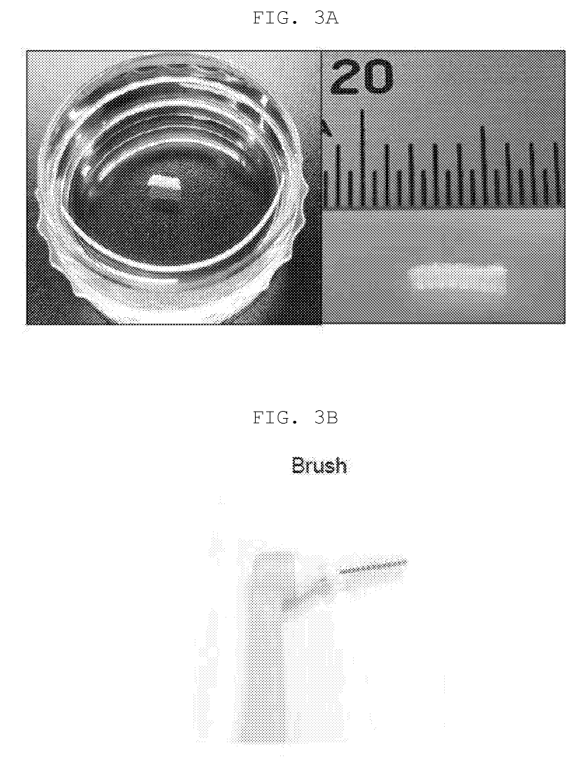

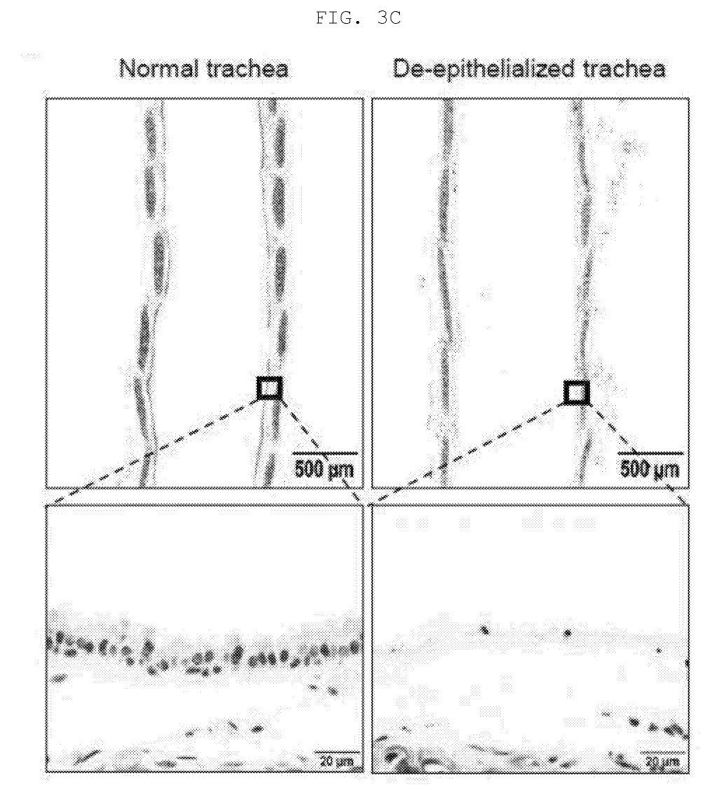

Total 36 C57BL/6 mice were used as recipient animals. The animals were divided into two groups having different post-operation periods (7 days and 14 days), and then sacrificed. The recipients were randomized to receive normal tracheas (n=12), de-epithelialized tracheas (n=12), or de-epithelialized tracheas having grafted human airway epithelia (n=12). The trachea of a C57BL/6 mouse was grafted in another C57BL/6 mouse according to the conventional method (Hua et al., 2010). The recipient animal was anesthetized with IP injection of Avertin (400 mg/kg, Sigma-Aldrich). At first, in order to visualize the whole laryngotracheal complex, a short midline cervical incision was conducted. After gently incision of the recipient organ, it was incised between the third cartilage below the epiglottis. Donor trachea segments were grafted using 9-0 Prolene suture threads in end-to-end starting from distal anastomosis (FIGS. 3A-3C).

The cervical incision part was sutured in the form of a layer using continuous 7-0 Vicryl suture threads (Ethicon), followed by suturing the skin with intermittent 7-0 Vicryl suture threads. Surgical procedures were performed in aseptic manner with assistance of a dissecting microscope. All recipient animals did not involve immuno-suppression.

### 5. Histological assessment

For histological and immuno-histochemical analysis, trachea grafts were harvested from the recipient mice on day 7 and day 17 after transplantation. Each trachea graft was cut into longitudinal sections for H&E staining or immuno-histochemical staining. Each graft piece was fixed in 4% formalin at room temperature for 24 hours. The formalin-fixed tissues were embedded in paraffin, followed by slicing the tissue into 4 μm sections.

For comparison of characteristics between groups, several different staining processes were performed: according to the instructions of manufacturers, in an aspect of general morphology, hematoxylin and eosin (H&E; Sigma-Aldrich), in terms of goblet cells, periodic Acid-Schiff (Sigma-Aldrich) staining, and Masson’s trichrome (Sigma-Aldrich) staining for reinforcing collagen deposition. The numbers of neutrophils, macrophages and goblet cells were indicated by an average number of four HPFs, respectively. Fibrosis was determined through Image J software (version 1.52p, NIH Image Processing Analysis). For assessment of a diameter of the organ lumen, it was measured at least three times at different points in desired region of each HPF, and Image J software was used for measurement.

### 6. Immuno-histochemical assessment of human airway epithelia

Immuno-histochemical analysis was performed by means of polink-2+ polymerized HRP wide-range DAB detection system (Golden Bridge International Labs., Cat. No. D41-18). Specifically, a paraffin section of nasal cavity tissues was mounted on a slide and dried at room temperature for 24 hours. The section was deparaffinized, re-hydrated, and autoclaved in 100 mml/L citrate buffer (pH 6.0; Dako) at 121° C for 10 minutes, thereby restoring an antigen. The antigen was treated with 3% hydrogen peroxide (H2O2) in methanol for 10 minutes, and then, the section was cultured in 3% BSA at room temperature for 1 hour, thereby blocking non-specific signals.

Successively, tissue sections were cultured along with antibodies to STEM101 (1:100; Y40400, Takara Bio), E-cadherin (1:100; #3195, Cell signaling), Ki-67 (1:100; ab15580, Abcam) and p63 (1:100; Cat No, Company) at 4° C overnight. After culturing the sections in a wide-range antibody enhancer and polymer-HRP, the cultured product was stained by the DAB detection system. In order to standardize coloring, a culturing time of diaminobenzidine stains was fixed in all experiments. The sections were contrast-stained with hematoxylin QS (Vector Laboratories Inc., Cat. No. H-3404) for 3 minutes, dehydrated through different grades of ethanol series, removed with xylene, followed by mounting using PERMOUNT^{TM} mounting medium (Fisher Scientific, Cat. No. SP15-100). A negative control was subjected to experiment while omitting the primary antibody, and all experiments have complied with the instructions of manufacturers unless otherwise specified. The slide was evaluated with an optic microscope (BX-51; Olympus) equipped with a camera (DP70; Olympus) in microscopic fields of view for wholly stained sections. Four regions of each tissue section were selected for assessment under high magnification field (HPF, magnification × 1000), and measurement was implemented by two inspectors.

### 7. Immuno-fluorescent staining

For immuno-fluorescent staining, 4 μm tissue sections were deparaffinized and rehydrated using ethanol washing series. The deparaffinized tissue sections were heated in citrate buffer (10 mM sodium citrate, pH 6.0, 0.05% Tween20) at 98° C for 10 minutes to perform epitope restoration. The section was washed with 1 × PBS three times for 5 minutes per time. The tissue section was pre-cultured in PBS containing 0.1% Triton x-100 and 3% BSA, followed by applying the corresponding primary antibody thereto and leaving the same at 4° C overnight. Next, the antibody (dilution ratio indicated) was used for immuno-staining experiments: E-cadherin (1:100; #3195, Cell signaling) and Ac-Tubulin (1:100; T6793, Sigma). Then, the tissue section was cultured in a mixture of Alexa-555 and Alexa-488 conjugate secondary antibodies diluted in a blocking buffer (1:400; Invitrogen) at room temperature for 2 hours. A nucleus was contrast-stained with 1 mg/mL DAPI (Sigma, Cat. No. D9542), and the slide was mounted on a fluorescent mounting medium (Vectashield, Vector Laboratories Inc., Cat. No. H-1000). All immuno-fluorescent staining processes were performed in a dark place, and images were acquired with Zeiss fluorescent microscope by AxioVision software (Carl Zeiss). In the control cultured with normal serum instead of the primary antibody, immuno-staining was not observed.

### 8. Statistic assay

By drawing Kaplan-Meier survival curve, survivals were compared between three groups. The comparison between the groups was implemented by non-parametric Mann-Whitney test. All data were represented by average ± SD wherein p < 0.05 is considered to be significant in the statistics. The illustrative figures were prepared using Prism version 8.2.0 (GraphPad Software Inc.) and SigmaPlot version 10.0 (Systat Software Inc.).

### 2. Experimental results

### 1. Animal survival after orthotropic tracheal transplantation

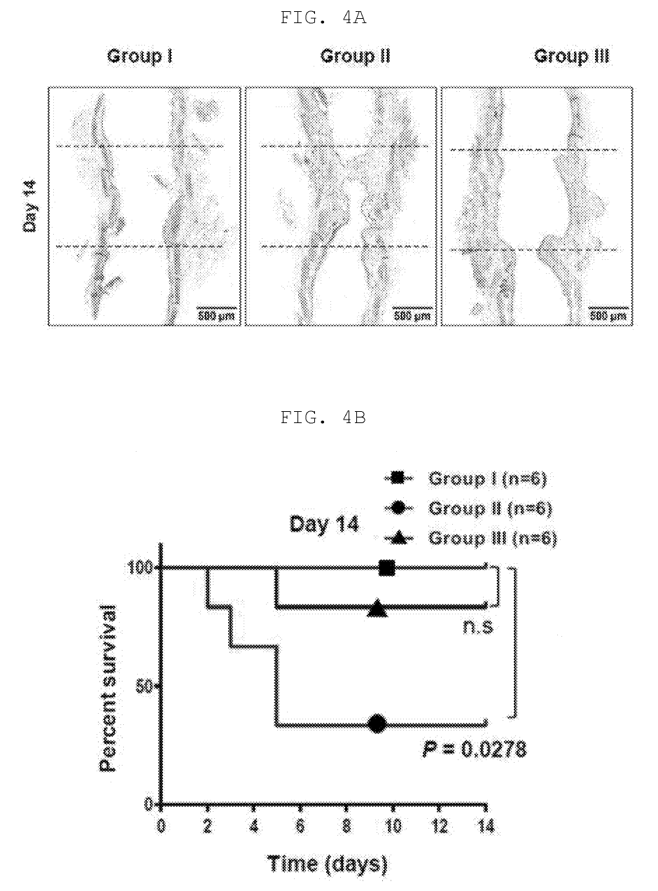

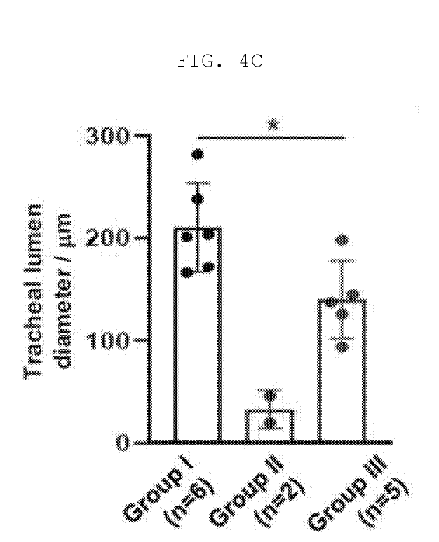

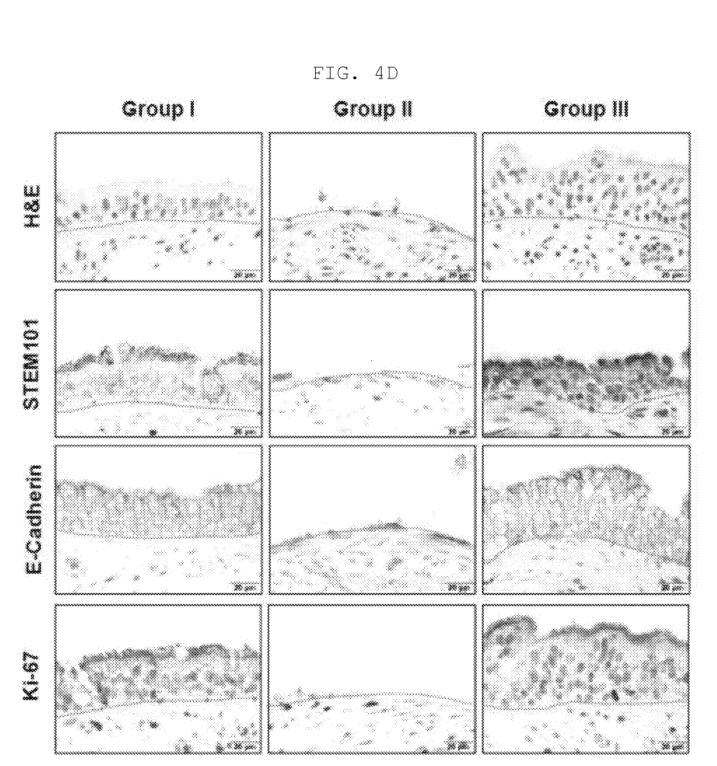

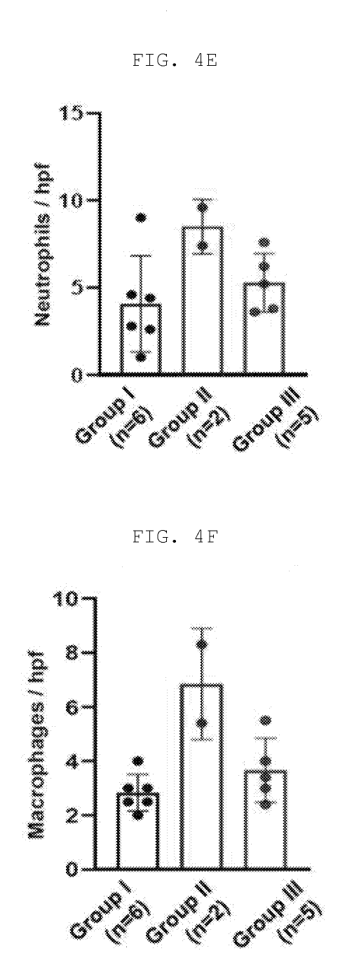

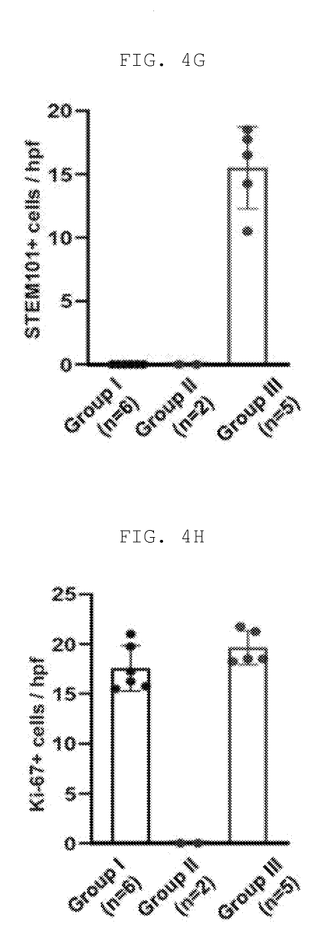

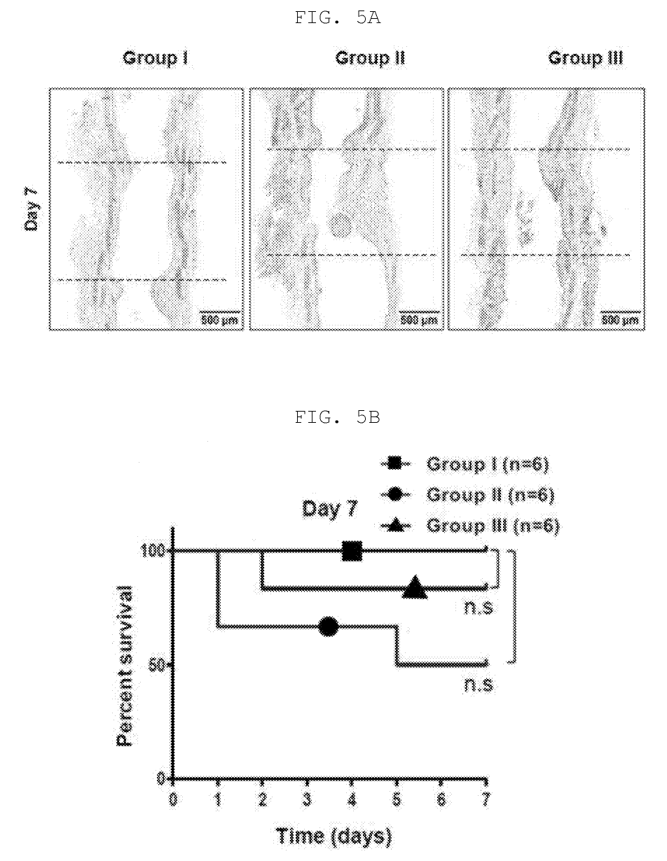

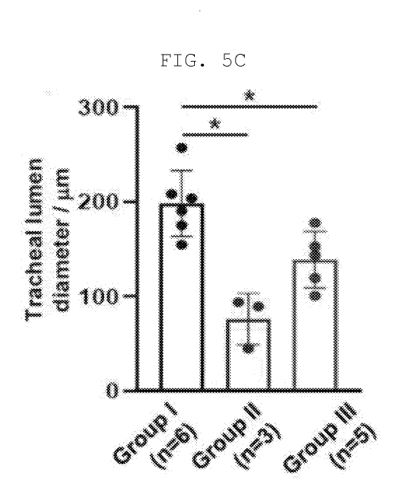

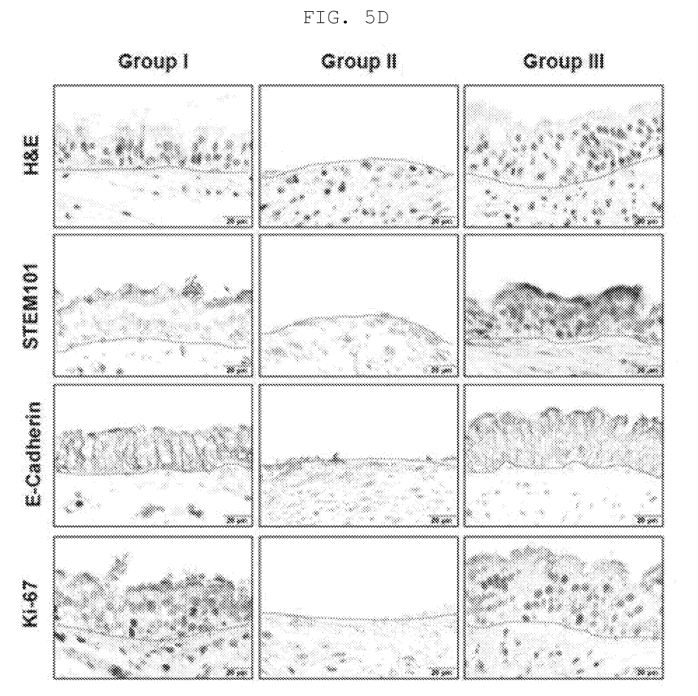

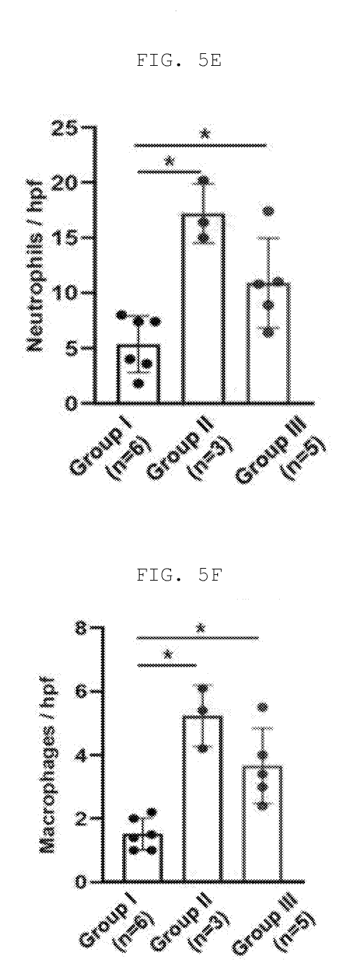

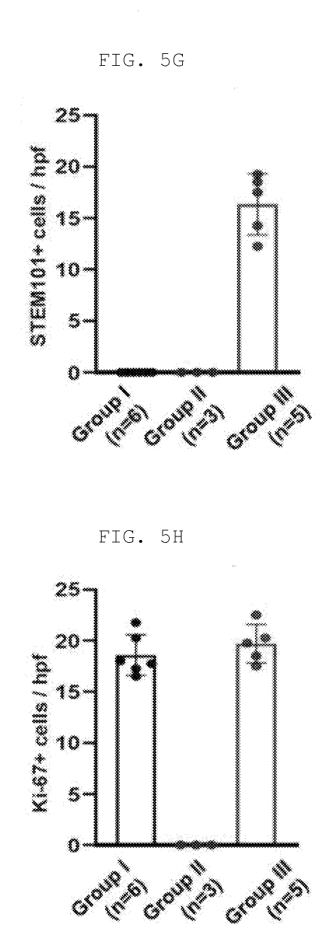

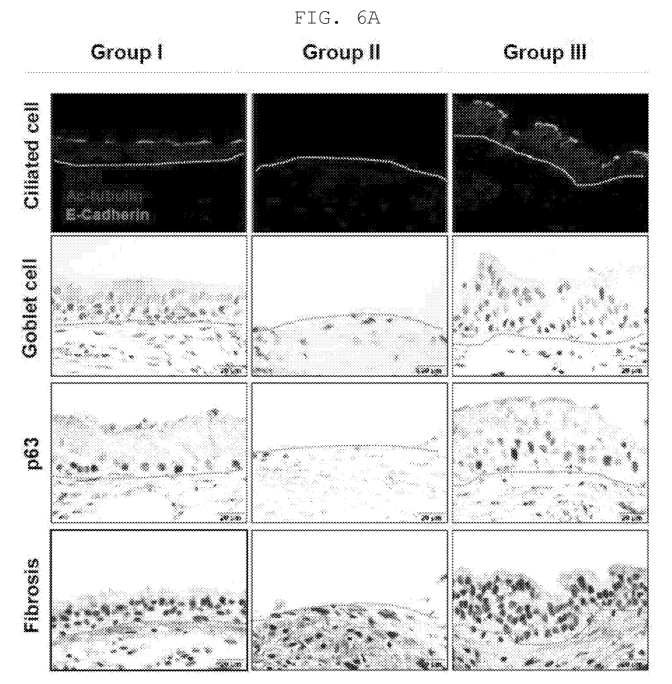

In the present study, trachea segments were collected for analysis on day 7 and day 14 after operation (FIGS. 2A and 2B). As a result of confirming H&E staining by a microscope, it was observed that the donor trachea was integrated into a host structure (FIG. 4A). All animals in group 1 were survived on day 7 and day 14. The observed tracheal lumen was not narrowed while a mucosa membrane of the trachea was similar to those of normal tracheas. In Group 2, 4 among 6 animals were dead within 14 days. The lumen of the de-epithelialized trachea was narrowed while observing a mass proliferation of granular tissues. In Group 3, 1 among 6 animals was dead after surgery, and a diameter of the tracheal lumen was considerably reduced compared to Group 1 (FIGS. 4A-4C and 5A-5C). These results suggest that the transplantation of human airway epithelia may have a possibility of improving the survival of an animal.

### 2. Histological observation of grafted human airway epithelia

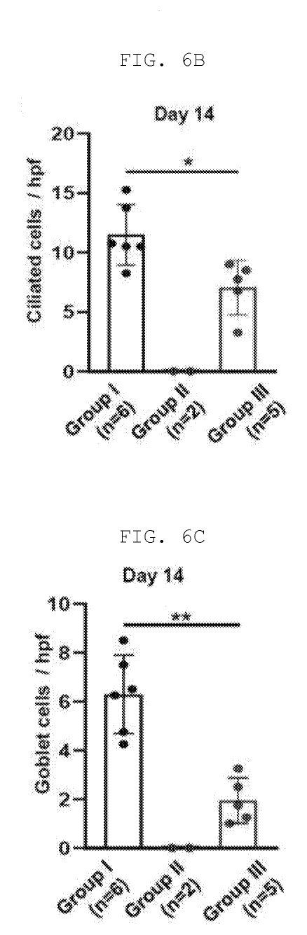

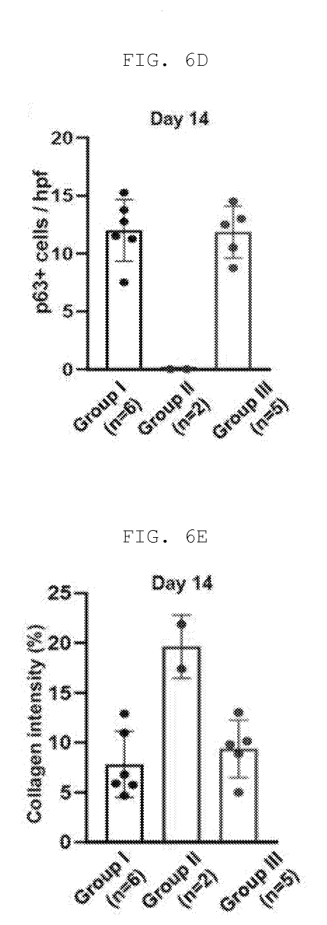

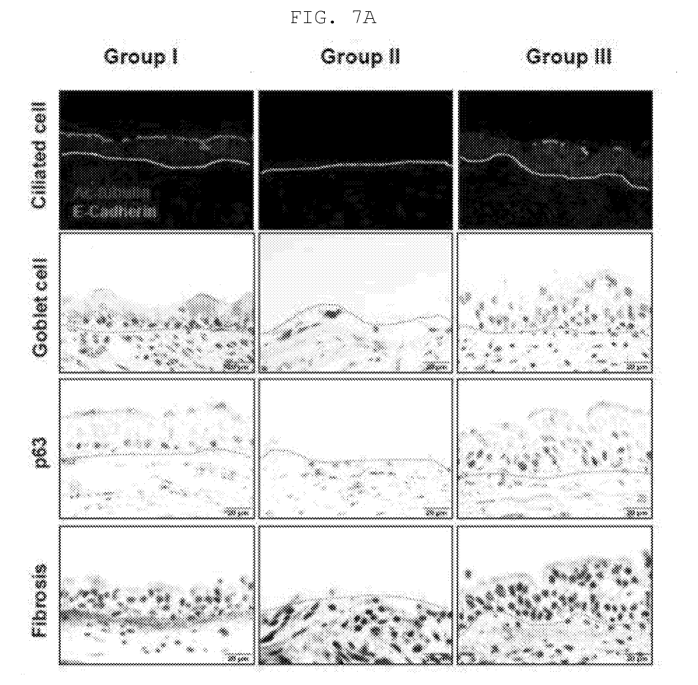

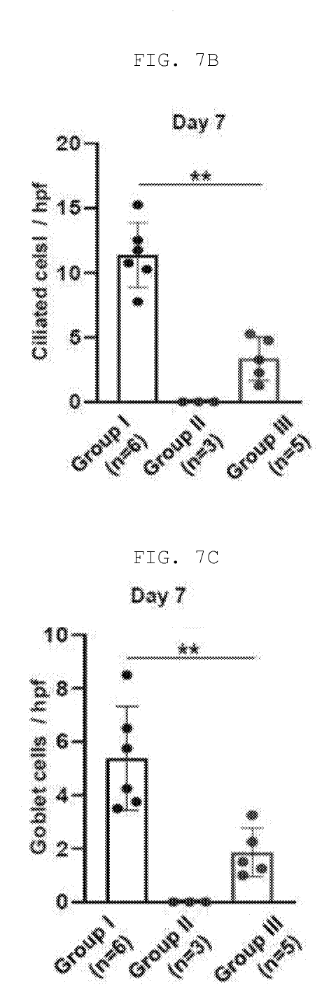

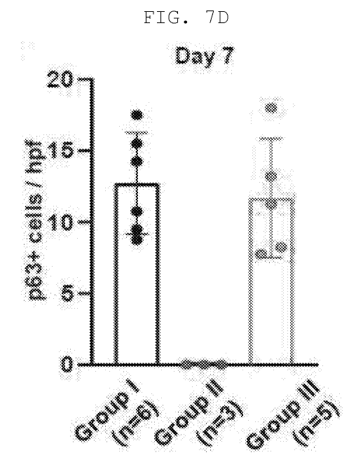

The existence of a whole epithelial surface could be found in all animals in Group 1. According to the immuno-histochemical analysis, E-cadherin (adhesive conjugate marker), Ki-67 (proliferation marker) and p63 (base cell marker) showed positive expression (FIG. 4D). Cilia cells and goblet cells were detected in the epithelia (FIGS. 6A-6C). By contrast, it was observed that regeneration of epithelia was absent due to severe inflammatory reaction to decrease the lumen of the de-epithelialized trachea (FIGS. 4D-4H and 5D-5H). The existence of human airway epithelia was confirmed in the grafted trachea for up to 14 days after operation by the optic microscope. The grafted human airway epithelia are composed of a thick layer (FIG. 4D). The immuno-histochemical analysis demonstrated positive expression of E-cadherin, Ki-67 and p63, while STEM101 (human nucleus marker) was detected in the epithelia of Group 3 only (FIGS. 6A-6B and 7A-7D). However, the number of cilia and mucus generating cells were significantly lesser than Group 1 on day 7 and day 14 (p < 0.01). In order to prove collagen deposition, the grafted tracheas, which were H&E stained and trichrome stained, were subjected to inspection using the microscope on day 14 after transplantation. Fibrosis was significantly found on the surface of the tubular lumen in the grafted trachea of Group 2 (FIGS. 6A and 6E). Further, trichrome staining of the grafted trachea in Group 3 demonstrated that collagen deposition under epithelia was smaller on Day 14 than Group 2. Therefore, the above results indicate that transplantation of human airway epithelia may have possibility of inhibiting inflammation and preventing collagen deposition.

What is claimed is:

1. A method for preparation of an epithelial cell tube, the method comprising:
(i) culturing epithelial cells at an air-liquid interface cell culture system to obtain differentiated epithelial cells;
(ii) transferring and adhering the differentiated epithelial cells as a layer to a planar support so that cilia of the epithelial cells face a lumen side; and
(iii) linking both ends of the planar support each other so that the layer of epithelial cells faces an outer peripheral surface of the planar support, resulting in forming the epithelial cell tube comprising a columnar support and the layer of differentiated epithelial cells adhered on the columnar support.

2. The method according to claim 1, wherein the culturing in the step (i) is performed by bringing cilia of the epithelial cells into contact with air while the bottom of the epithelial cells is exposed to a culture medium.

3. The method according to claim 1, wherein the layer of differentiated epithelial cells in the step (ii) is formed by adhering the differentiated epithelial cells to the planar support through electrostatic attraction.

4. The method according to claim 1, wherein the planar support is a porous membrane made of polyvinylidene difluoride (PVDF), polypropylene, polyethylene, cellulose or derivatives thereof, chitin, chitosan, collagen, urethane, or three-dimensional scaffold materials prepared using a 3D-printing technique.

5. The method according to claim 1, wherein the tube is formed by rolling the planar support carrying the layer of differentiated epithelial cells into a tubular shape and linking both ends of the planar support together, such that the layer of differentiated epithelial cells faces outward on the outer peripheral surface of the columnar support.

6. The method according to claim 1, wherein the layer of differentiated epithelial cells comprises pseudostratified epithelium with cilia and goblet cells while exhibiting polarity.

7. The method according to claim 1, further comprising the step (iv) of additional culturing the epithelial cell tube.

8. The method according to claim 7, wherein the additional culturing in the step (iv) is performed at an air-liquid interface cell culture system after the linking.
